(19) United States
(12) Patent Application Publication
(10) Pub. No.: US 2002/0103485 A1
(43) Pub. Date: Aug. 1, 2002

(54) Electrosurgical pencil with a smoke evacuating blade

(76) Inventors: Ivan Melnyk, Coquitlam (CA); John Kidder, Vancouver (CA)

Correspondence Address: Ivan Melnyk, #250-13155 Delf Place, Richmond, BC, V6V 2A2, CA

(21) Appl. No.: 09/729,310

(22) Filed: Dec. 5, 2000

(51) Int. Cl. A61B 18/18

(52) U.S. Cl. 606/45

(57) ABSTRACT

An electrosurgical pencil with a smoke evacuating blade to be used in general surgery and laporoscopy. The blade has a plurality of lateral holes and at least one longitudinal hole in its thicker central part. In a place where the blade is attached to a hadpiece, it is connected to a smoke evacuating tube that is pulled through the handpiece. The tube is inserted in a cable assembly together with electrical wires and connected to an vacuum source; the air flows through the blade to the tube. Due to close proximity to the cutting area, minimum negative pressure is required for sucking of smoke and no obscure of surgeon's view occurs.

### BACKGROUND OF THE INVENTION

1. Field of the Invention

This invention pertains to electrosurgery, and more particularly to a new and improved handpiece, or electrosurgical pencil, which incorporates a blade by which electrical energy is applied to the tissue and which serves as a nozzle for sucking the smoke from a treatment zone.

2. State of the Art

Electrosurgical treatment of tissue accompanies with generation of substantial amount of smoke which consists of carbonized tissue, blood, viral particles, DNA, bacteria, water, carbon dioxide and toxic gases. Recent medical regulations require to have smoke evacuators whenever tissue is vaporized. A number of smoke evacuators have been proposed since introducing such requirements.

The very first smoke evacuators included a tube connected to a source of vacuum which run parallel to the cautery blade. Such arrangement is disclosed, for example, in U.S. Pat. Nos. 3,906,955, and 4,362,160. Electrosurgical instruments utilizing this approach effectively remove smoke; however, the nozzle of the sucking means obstructs the surgeon's view of the surgical site.

Other patents, such as U.S. Pat. Nos. 3,974,833; 4,562,838; 4,683,884; 4,719,914, and 5,181,916 disclose an electrosurgical instrument with a smoke sucking nozzle which is concentric with the cutting blade. The efficiency of evacuating smoke by such concentric designs is low because smoke is generated at the distal end of the blade (where blade is applied to the tissue) and it is sucked through the nozzle which is located at the proximal end of the blade (where blade is assembled with a handpiece and connected to the electrical wire). The typical electrosurgical blade is about 2 cm long; as a result, the negative pressure around the narrow nozzle that is achieved by an aspirator will be insufficient to evacuate smoke remotely. The smoke evacuating efficiency can be improved by increasing the power of the aspirator. However, the increase of vacuum power produces a constant intense noise when the electrosurgical instrument is used. Turning the air flow in a proximity of the nozzle as it is disclosed in U.S. Pat. Nos. 5,431,650 and 5,460,602 (vortex shape of the nozzle) does not improve the remote evacuating efficiency due to rapid pressure dissipation along the blade.

### SUMMARY OF THE INVENTION

It is therefore an object of the invention to provide an electrosurgical pencil with a smoke evacuator which can effectively remove the smoke from the surgical site.

It is also an object of the invention to provide an electrosurgical pencil with a smoke evacuator which will not obscure the view of the surgical site.

It is further an object of the invention to provide an electrosurgical pencil with a smoke evacuator which does not require increase of vacuum power, and thus, which does not produce the constant intense noise during the surgery.

According to the present invention, the sucking nozzle of the smoke evacuating system is located directly on the cutting blade of the electrosurgical pencil. In one embodiment of the electrosurgical pencil, the nozzle is made as a plurality of lateral holes that are connected to a main longitudinal hole. The main longitudinal hole is coupled to a smoke evacuating tube that is enclosed in the pencil's body; its distal part is connected to the aspirator. Due to location of the nozzles very close to the smoke generating area, the smoke is effectively evacuated without spreading far away. In another embodiment of the electrosurgical pencil, the electrosurgical blade has an empty space in its central portion which is covered with two from both sides. The brackets have holes or grooves, thus being applied to the flat blade surface of the blade brackets create pluralities of nozzles that are located very close to the cutting zone.

### BRIEF DESCRIPTION OF THE DRAWINGS

FIG. 1 is a view of a first embodiment of the electrosurgical smoke evacuating blade of the invention;

FIG. 2 is a cross section of the blade of FIG. 1;

FIG. 3 is a cross section of the blade of FIG. 1 connected to a smoke evacuating tube and a power wire;

FIG. 4A is a top view of the cross section of the blade of FIG. 1 with through hole lateral holes;

FIG. 4B is a top view of the cross section of the blade of FIG. 1 with sequencing lateral holes.

FIG. 5 is a cross section of the blade of FIG. 1 with lateral holes of different diameters;

FIG. 6 is a schematic view of the location of the blade of FIG. 5 against the tissue;

FIG. 7 is a schematic three-dimensional view of the electrosurgical pencil with a smoke evacuating blade of the invention;

FIG. 8A is a cross section view of the cable assembly of the electrosurgical pencil of FIG. 7 with two electrical switching wires;

FIG. 8B is a cross section view of the cable assembly of the electrosurgical pencil of FIG. 7 with an optical fiber for switching operation modes of the pencil;

FIG. 9A is a schematic view of the disassembled second embodiment of the electrosurgical smoke evacuating blade of the invention;

FIG. 9B is a view of the assembled electrosurgical smoke evacuating blade of FIG. 9A;

FIG. 10A is a schematic view of the disassembled another embodiment of the electrosurgical smoke evacuating blade of the invention with vertical grooves on the brackets;

FIG. 10B is a view of the assembled electrosurgical smoke evacuating blade of FIG. 1A;

### DETAILED DESCRIPTION OF THE INVENTION

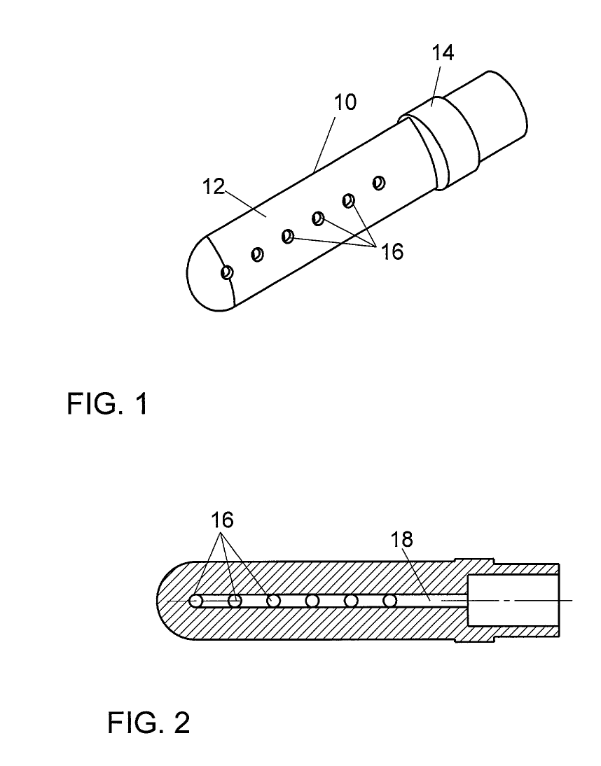

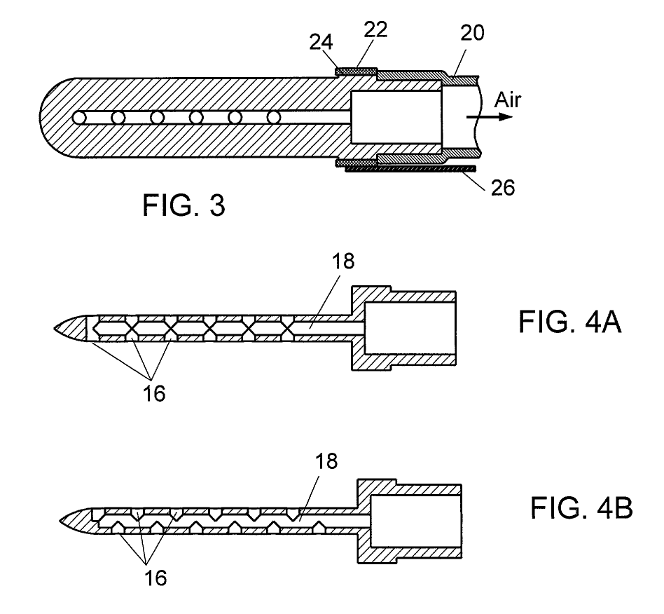

An example of the electrosurgical blade with a sucking nozzle on it according with the present invention is shown in FIG. 1. The blade (10) includes a cutting part (12) and a holding part (14). Preferably, the cutting part has shape and size the same as that of prior art, such as of elliptical cross section and rounded edges. The holding part is preferably cylindrical; it serves for holding the blade in the pencil and connecting the blade to a power wire as it is explained below. A plurality of lateral holes (16) are made along the thicker central area. They are connected to a longitudinal hole (18) which is made along the blade in its thicker part. The longitudinal hole (18) is coupled to a smoke evacuating pipe (20) as is shown in FIG. 3. Preferably, the pipe (20) is elastic so the coupling is done by inserting the holding part of the blade into the pipe. Another end of the pipe is connected to an aspirator (not shown in FIG. 3). A ring electrode (22) is located at the distal end of the pencil where the blade is mounted. It is contacted with a collar (24) of the blade; preferably, this contact is a common spring-loaded contact that is typically used in electrosurgical pencil. The collar (24) limits the insertion of the blade into the pipe. The ring electrode is connected to a power wire (26) that is connected do a high frequency electrosurgical generator.

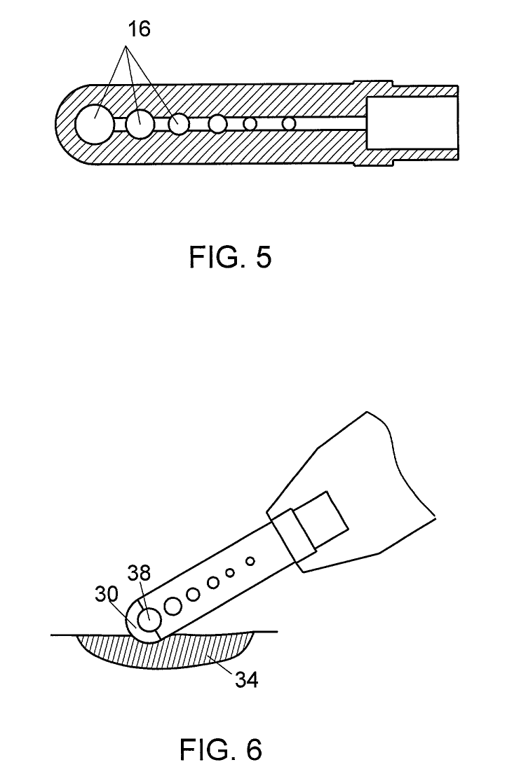

The lateral holes (16) can be made through the blade as is shown in FIG. 4A, or they can be made sequentially at each side of the blade as is shown in FIG. 4B. A sequential design is preferable due to more uniform distribution of the negative pressure along the blade. The size of the lateral holes may vary from larger holes at the distal end of the blade to smaller holes at the proximal end of the blade as is shown in FIG. 5. This variation of the size will also provide more uniform distribution of the sucking (negative) pressure by compensation the air resistance in small holes. The blade is typically applied under certain angle to the tissue, as shown in FIG. 6. The distal end (30) of the blade is inserted deeper in the tissue (34) than the central cutting part (36) of the blade. Consequently, much more smoke is generated from the distal end and less smoke is generated from the central part of the blade. The first lateral hole (38) can be much bigger than the following lateral holes in order to suck more smoke from the distal end of the blade.

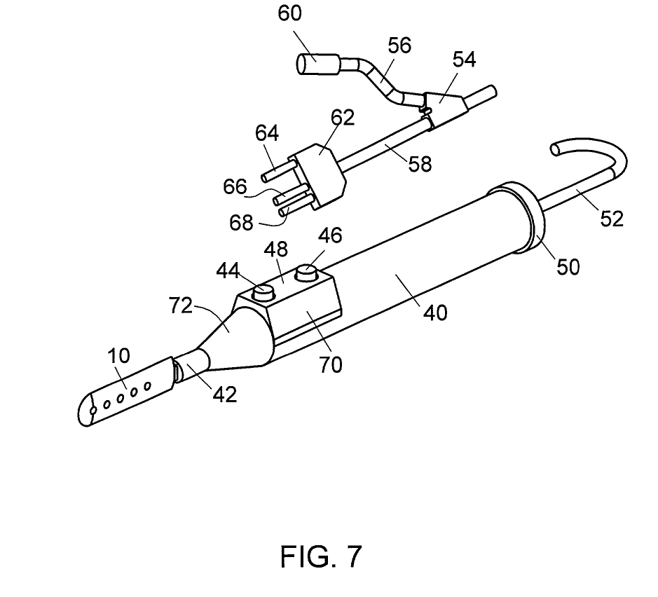

A schematic general view of the electrosurgical pencil according with the present invention is shown in FIG. 7. The pencil includes a body (40) with a narrower neck (42) where the blade (10) is inserted. Buttons (44) and (46) are common for electrosurgical pencil; they are used to finger activate a switch (48) which changes the mode of operation, typically “Coagulation” and “Cutting” mode. The switch is permanently “Off” if no button is pressed. A proximal end (50) of the body holds a cable assembly (52) which includes a smoke evacuating tube, electrical wires and an elastic jacket holding these items together. The cable assembly is sufficiently long to operate in a surgical room; typically, it is from 2 to 3 m long. The cable assembly has a splitter (54) at its distal end which separates the evacuating tube (56) from the electrical cable (58) which includes all electrical wires of cable assembly (52). The tube ends by a vacuum plug (60) that is connected to an aspirator (not shown). The electrical cable has an electrical plug (62) with typically three contacts (64), (66), and (68). The contact (64) is general; it is directly connected to the blade (10). Contacts (66) and (68) are connected to the switch (48), they connect the output of the electrosurgical generator (not shown) to the contact (64) depending on the mode of operation, either, “Coagulation”, or “Cutting”.

The body (40) can be designed in a variety of ways in order to get firm gripping by the operating surgeon. For instance, it can have a flat side portion (70) and a conical part (72) that connects the body (40) and the neck (42). The switch (48) can be a typical electrical microswitch connecting to the electrical plug (62) with two electrical wires; or it can be a single fiber optical switch such as disclosed in U.S. Pat. No. 5,8982,862. In case of optical switch, a single optical fiber is enclosed into the cable assembly and an optical connector at the distal end of the cable assembly is plugged to an adapter (not shown) that activates the contacts (66) and (68).

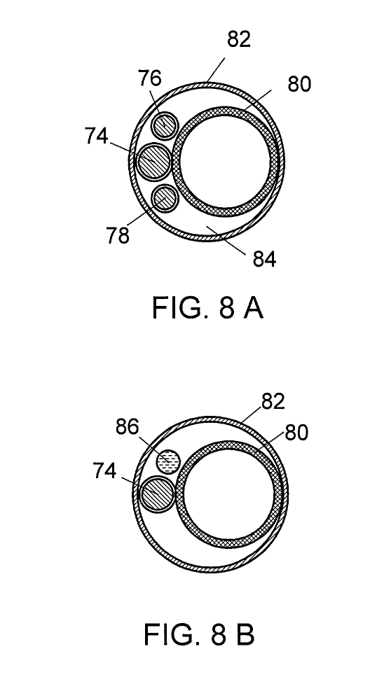

The cross sections of the cable assemblies with electrical wires and optical fiber are shown in FIG. 8A and FIG. 8B, accordingly. A power wire (74) is accompanied by two switching wires (76) and (78), the switching wires are typically thinner as they do not carry high power. The thickness of the cable assembly is mostly determined by a size of the smoke evacuating tube (80) which inner diameter is ranged from 1.0 to 10 mm, preferably from 3.0 to 6.0 mm. The wires and the tube are hold together by a jacket (82); a space (84) between the wires and the tube can be filled with an elastic material, such as silicone or rubber. The optical fiber (86) in FIG. 8B is preferably a plastic fiber made of acrylic (PMMA), polycarbonate, polystyrene or another optically transparent material. The diameter of the fiber is ranged from 0.1 to 0.5 mm, preferably 0.25 mm.

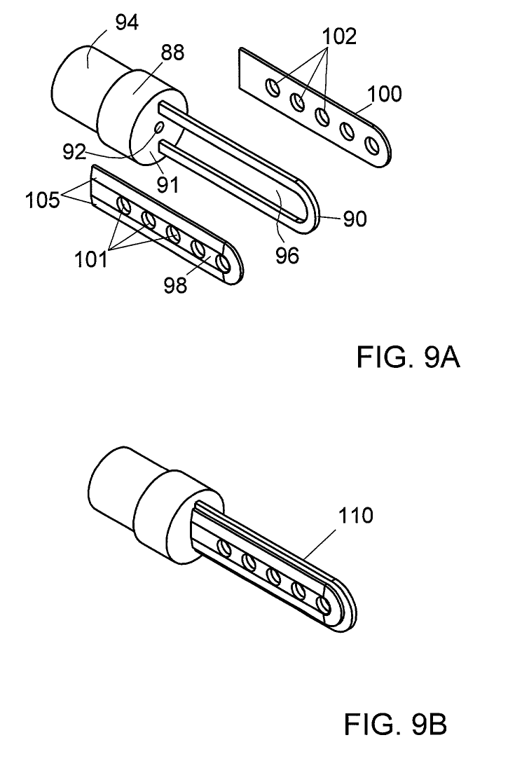

Referring now to FIG. 9A another embodiment of the electrosurgical sucking blade is disclosed. The blade includes a metallic base (88) with a thin metallic plate (90) on its front part (91) and a smoke evacuating hole (92). The lateral size of the thin plate is the same as a typical elecrosurgical blade, the length is from 10 to 30 mm, the thickness is from 0.1 to 0.5 mm, the width is from 2.5 to 5 mm. A narrower part (94) of the base is connected to the evacuating tube (not shown) whereas a thicker part of the blade can be connected to the power wire. The thin plate (90) has an open space (96 in its central part. This open space serves as a longitudinal hole (18) as was shown in FIG. 2. Two brackets (98) and (100) are attached to both sides of the thin plate. The brackets have pluralities of holes (101) and (102). These holes serve as lateral holes (16) as was shown in FIG. 2. The brackets have sharpened peripheral edges (105); they are narrower than the thin plate, thus being applied to the thin plate sharp edges (110) of the plate remains open as shown in FIG. 9B.

The thin plate (90) is made of the same metal as the base (88), typically of stainless steel or titanium alloy. High frequency electrical power is delivered to the tissue from the base through the sharp edges (110) of the plate where cutting or coagulation is occurred. The brackets (98) and (100) can be made of the same metal or they can be made of a non-conductive high temperature stable material, such as Teflon, for example. Teflon is known for its low adhesion to heated organic substances, therefore, no periodic cleaning of the blade will be required during the surgery.

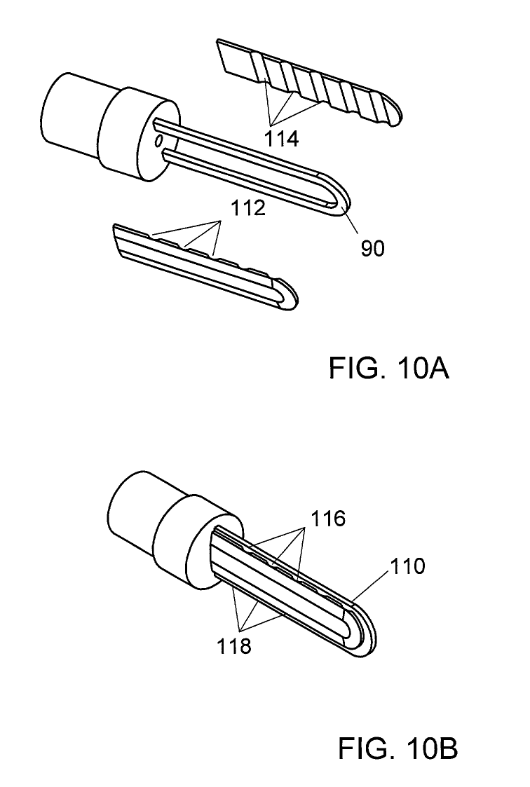

As is shown in FIG. 10A, the brackets can be made with pluralities of vertical grooves (112) and (114) instead of holes. Being applied to the thin plate (90), the grooves create rows of smoke evacuating nozzles (116) and (118) that are located at both sides of blade close to the plate edge (110) as is shown in FIG. 10B. The location of the nozzles close to the cutting area creates better conditions for evacuating smoke during the surgery because smoke will not far spread yet and consequently it can be evacuated with much lower negative pressure and lower air flow. It is understandable for those skilled of the art, a combination of holes (shown in FIG. 9A) and grooves (shown in FIG. 10A) can be utilized for better evacuation of the smoke.

Electrosurgical blades described above provide better conditions for smoke evacuation during the electrosurgical procedures due to very close location of the sucking nozzles to the smoke generating area. Because smoke evacuation may be achieved with a low air flow, the smoke evacuating tube can be thin enough to be placed together with electrical wires into the cable assembly. The thin smoke evacuating tube can be easily placed into existing electrosurgical pencil's geometry and connected to the blade. The electrosurgical blades with smoke evacuating nozzles do not obscure the surgeon's view as they have the same size as typical electrosurgical blades.

### ELECROSURGICAL PENCIL WITH A SMOKE EVACUATING BLADE

References Cited

U.S. PATENT DOCUMENTS

| U.S. Pat. No. | Issued | Inventor(s) | Title | Class |
|---|---|---|---|---|
| 3906955 | 09/1945 | Roberts | Surgical cauterizing tool having suction | 128/303. |
| | | | means | 17 |
| 3974833 | 08/1976 | Durden | Disposable electrosurgical cautery | 604/020 |
| | | | having optional suction control feature | |
| 4161950 | 07/1979 | Doss et al. | Electrosurgical knife | 128/303. |
| | | | | 14 |
| 4362160 | 12/1982 | Hiltebrandt | Endoscopes | 128/303. |
| | | | | 15 |
| 4562838 | 01/1986 | Walker | Electrosurgery instrument | 128/303. |
| | | | | 14 |
| 4683884 | 08/1987 | Hatfield et al. | Noise attenuating smokeless surgical | 128/303. |
| | | | device | 14 |
| 4688569 | 08/1987 | Rabinowitz | Finger actuated surgical electrode | 128/303. |
| | | | holder | 14 |
| 4719914 | 01/1988 | Johnson | Electrosurgical instrument | 128/303. |
| | | | | 1 |
| 5015243 | 05/1991 | Schifano | Means for removing smoke from a | 604/315 |
| | | | operative site | |
| 5181916 | 01/1993 | Reynolds et al. | Surgical probe and smoke eliminator | 606/16 |
| 5226904 | 07/1993 | Gentelia et al. | Electrosurgical instrument | 606/42 |
| 5256138 | 10/1993 | Burek et al. | Electrosurgical handpiece incorporating | 606/42 |
| | | | blade and conductive gas functionality | |
| 5269781 | 12/1993 | Hewell | Suction-assisted electrocautery unit | 606/405 |
| 5413575 | 05/1995 | Haenggi | Multifunction electrocautery tool | 606/45 |
| 5431650 | 07/1995 | Cosmescu | Vortex hand piece shroud for automatic | 606/41 |
| | | | smoke evacuator system for a surgical | |
| | | | laser apparatus and method therefore | |
| 5460602 | 10/1995 | Shapira | Smoke evacuator for smoke generating | 604/22 |
| | | | devices | |
| 5892862 | 04/1999 | Kidder | Flexible mirror optical switch | 385/16 |
| 6063083 | 05/2000 | Duong-Van | Electrosurgery blade having discrete | 606/45 |
| | | | point discharge saw-tooth edge | |
| 6099525 | 08/2000 | Cosmescu | Removable shroud for receiving a | 601/41 |
| | | | pencil used in electro-surgery | |

FOREIGH PATENT DOCUMENTS

| | | | | |
|---|---|---|---|---|
| EP 0280798 | 09/1988 | European Pat. | Electrosurgery surgical instrument | A61B17/39 |
| | | Off | | |

OTHER PUBLICATIONS

Winstin C., The effects of smoke plume generated during laser and electrosurgical procedures. Min. Inv. Surg. Nursing, 1994, v.3, p.8.

O'Grady K F, et al., Electrosurgery smoke: hazards and protection. J. Clin. Eng., 1996, v.3, p.21.

Roming C L, et al., Regulation of surgical smoke plume. AORN Journal, 1997, v.4, p.65.

What is claimed is:

1. An electrosurgical pencil with a smoke evacuating blade comprising:
a handpiece with a switching means for selection the operation mode;
at least one power wire that is inserted into said handpiece;
an electrosurgical blade that is connected to a distal end of said handpiece;
a smoke evacuating means;
a cable means that are inserted into proximal end of said handpiece.

2. The electrosurgical pencil of claim 1, wherein:
said electrosurgical blade is thicker in its central part and sharper at its edges;
said electrosurgical blade has a plurality of lateral holes and at least one longitudinal hole that is made in said thicker central part;
said longitudinal hole is connected to all said lateral holes;
said electrosurgical blade is connected to said power wire in a distal end of said handpiece.

3. The electrosurgical pencil of claim 1, wherein:
said smoke evacuating means comprising an elastic smoke evacuating tube;
said smoke evacuating tube is pulled through said handpiece and is inserted into said cable means;
a proximal end of said smoke evacuating tube is split from said cable means and is connected to a vacuum source.

4. The electrosurgical pencil of claim 1, whereins:
said electrosurgical blade has a prolonged proximal part that is inserted into the distal end of said handpiece and is connected with a distal end of said smoke evacuating tube;
said connection provides a possibility for air to flow through said lateral holes, said longitudinal hole, said smoke evacuating tube to said vacuum source.

5. The electrosurgical pencil of claim 2, wherein
diameters of said lateral holes are from 0.5 to 5.0 mm;
diameter of said longitudinal hole is from 0.5 to 2.5 mm.

6. The electrosurgical pencil of claim 2, wherein said lateral holes have different diameters.

7. The elctrosurgical pencil of claim 6, wherein said lateral holes are larger at the distal end of said electrosurgical blade.

8. The electrosurgical pencil of claim 4, wherein:
said electrosurgical blade has a thin flat distal portion with an empty space in a center that provides the air flow to said smoke evacuating tube;
said lateral holes are made in brackets that are attached along both sides of said thin flat distal portion;
said brackets are from 0.5 to 2.0 mm narrower than said thin flat distal portion;
said lateral holes in said brackets provides air flow to said empty space in said thin flat distal portion;

9. The electrosurgical pencil of claim 8, wherein said brackets are made of the same material as said electrosurgical blade.

10. The electrosurgical pencil of claim 8, wherein said brackets are made of thermally resistant plastic.

11. The electrosurgical pencil of claim 10, wherein said plastic is Teflon.

12. The electrosurgical pencil of claim 4, wherein:
said electrosurgical blade has a thin flat distal portion with an empty space in a center that provides the air flow to said smoke evacuating tube;
two brackets are applied to both sides of said thin flat distal portion;
said brackets are from 0.5 to 2.0 mm narrower than said thin flat distal portion;
said brackets have vertical grooves on the sides that are contacted said thin flat distal portion;
said vertical grooves in said brackets provides air flow to said empty space in said thin flat distal portion;

13. The electrosurgical pencil of claim 12, wherein said brackets are made of the same material as said electrosurgical blade.

14. The electrosurgical pencil of claim 12, wherein said brackets are made of thermally resistant plastic.

15. The electrosurgical pencil of claim 14, wherein said plastic is Teflon.
